(19) United States
(12) Patent Application Publication
(10) Pub. No.: US 2022/0031905 A1
(43) Pub. Date: Feb. 3, 2022

(54) Personal Space Germicidal Air Sterilizer And Methods

(71) Applicants: John McEllen, Chagrin Falls, OH (US); L. Lawton Rogers, III, Georgetown, SC (US)

(72) Inventors: John McEllen, Chagrin Falls, OH (US); L. Lawton Rogers, III, Georgetown, SC (US)

(73) Assignee: MYLIGHT LLC, Georgetown, SC (US)

(21) Appl. No.: 16/945,565

(22) Filed: Jul. 31, 2020

(51) Int. Cl. A61L 9/20

(52) U.S. Cl. CPC A61L 9/20; A61L 2209/10

(57) ABSTRACT

A desktop personal breathing space UV-C air sterilizer in which sterilized air from the top of the sterilizer is adjustably directed to the space from which an individual inhales, and in which ai exhaled by the individual air is mixed with air from the general area of the individual and drawn into the bottom of the UV-C air sterilizer across a horizontally disposed UV-C source, to provide a directional stream of sterilized air to the personal breathing space of the individual. Methods are disclosed.

### BACKGROUND OF THE INVENTION

The present invention relates to an improved device for using ultra violet radiation to reduce the concentration of airborne germs within the breathing space of an individual.

The threat of pandemic is ever present and include recurring viruses [such as influenza and rhinovirus (the common cold)], sporadic viruses [such as H1N1 (Swine Flu), Bird Flu, SARS Covid 2 and the derivative SARS Covid 19], bacteria [such as strep, c diff and staph], spores caused by mold or anthrax, and bio-terrorist threats. All of these present ongoing, if not constant, health dangers. The process by which these germs are spread varies and social distancing is helpful only with respect to heavier droplets which fall by gravity in a very short distance, i.e., one to two meters.

However, the exhalation of aerosolized germs is also considered to be a means by which a virus is spread, i.e., a contagious person may continually contaminate the air within a room by the act of breathing, coughing, singing or shouting. This contaminated air is circulated within the room and throughout the building by air currents, with aerosolized particles remaining in the air reportedly between 2 and 16 hours for particles less than 0.5 microns.

With aerosolized germs suspended or “floating” in the air, the relationship of germ concentration to contagion is not well understood across the broad spectrum of viruses. For some pathogens, the predominant form of transmission is by aerosolized germs. However, it is generally accepted that a reduction in the concentration of germs in the breathing space of an individual is beneficial as is limiting the time of exposure, i.e., contagion is a function of both germ concentration and time of exposure.

In many areas, the wearing of masks is mandated but it is known that aerosolized virus cells are 0.3 microns in diameter and that even N95 medical masks filter only particles 0.5 microns or larger. Recent studies suggest that the SARS Covid 19 virus is also transmitted in suspended airborne particles of <0.5 microns.

It has long been proposed to kill airborne germs by circulating the air through a killing field of confined UV-C irradiation. This process has been proposed for rooms of varying size, i.e., auditoriums and building lobbies, hospital wards and conference rooms, and individual offices and doctor's examining rooms. Large upper room sterilizers are useful with large gatherings where the volume of air is large.

Portable ai purifiers with a UV-C component are so small that the area influenced by the unit is often trivial relative to the size of the room. An example of such a small entire room sterilizer may be found in McEllen et al US 2007/0036696 where air is disclosed as taken into the unit at the bottom thereof, sterilized within the unit, and omnidirectionally expelled from the top of the unit into the room. The level of available germicidal protection of the breathing space of an individual working within such a room, e.g., an individual working at his desk or meeting with a second individual at a small conference table, is generally the same level of protection available to anyone at any place in the room. Over time, the air in the entire room is treated as it circulates through the unit, but such small volume entire room sterilizers generally have no significant impact on the personal breathing space of an individual in the room.

Efforts have been made to focus the protection on a small pocket of air within the room as a whole, so that an individual may locate himself within that small pocket of sterilized air and more quickly receive the germicidal benefits of the unit in what may be considered his personal breathing space.

McEllen US 2004/0184949 discloses a UV-C sterilizer that attempts to focus the germicidal benefits on a pocket of air, herein referenced as the “bubble”, within a room by discharging sterilized air into the breathing space of an individual. While this was advantageous over “whole” room sterilizers in reducing the concentration of germs in an individual's personal breathing space, the McEllen personal breathing space sterilizer has not proven to be effective for a number of reasons.

For example, the UV-C source disclosed by McEllen for use in a personal breathing space air sterilizer is a conventional single ended, elongated twin tube lamp mounted base up within a vertical passageway. The vertical orientation of the UV-C source requires that the length of the passageway, i.e. the vertical spacing between air input and air output apertures, be from about 18 to about 24 inches which, as discussed infra, creates a number of problems in sterilizing the air in a personal breathing space.

Moreover, the base up configuration of the source locates the cathode of the source at the point of greatest heat as the air within the passageway is heated by the source leading to a shortened source life.

In addition, the exhaustion of sterilized air from the base of the unit whose contemplated location is on a table or desk greatly increases the likelihood of interference with the passage of air from the output aperture to the personal breathing space by objects usually found on the desk.

Finally, the broad spread in the direction in which McEllen exhausts sterilized air, i.e., an arc spanning from about 60 degrees to about 120 degrees, coupled with the reduced air velocity, significantly reduces the volume of sterilized air that actually reaches the personal breathing space.

It is accordingly an object of the present invention to provide an improved air sterilizer for materially reducing the germ concentration in the breathing space of a single, perhaps two or three, individuals in a very specific location within a room, as contrasted with germ concentration reduction in an entire room, and improving known methods of so doing.

Another object of the present invention to provide a desktop personal space air sterilizer in which the delivery of sterilized air to the personal breathing space is materially improved.

Still another object of the present invention is to provide a small, lightweight, desktop personal breathing space sterilizer which applies the best features of small unit, whole room sterilizers to a personal breathing space sterilizer.

These and many other objects and advantages will be apparent from the following detailed description of preferred embodiments when read in conjunction with the appended drawings.

### THE DRAWINGS

FIG. 1 is a pictorial representation of an individual protected by the personal space germicidal air sterilizer of the present invention.

FIG. 2 is an exploded view of the housing of the air sterilizer of the present invention illustrating the creation of the personal breathing space.

FIG. 3 is a top plan view of the lower or sterilization chamber of the personal space germicidal air sterilizer of the present invention.

FIG. 4 is an elevation taken through lines 4-4 of FIG. 3 illustrating the sterilizing of the air entering the air sterilizer of the present invention.

### DETAILED DESCRIPTION OF PREFERRED EMBODIMENTS

With referenced to the drawings schematically illustrating the structure and operation of the air sterilizer of the present invention, like elements have been given like numerical designations throughout the drawings.

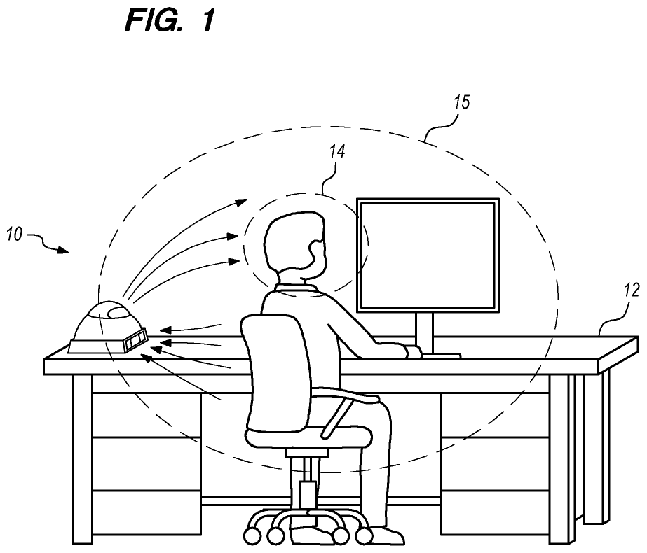

One embodiment of the personal breathing space desktop air sterilizer 10 of the present invention is pictured in FIG. 1 on a desk 12 within a much larger room, e.g., an open plan office. A single individual is seated at the desk 12 and the personal breathing space of the individual is the relatively small space from which the individual effectively draws air into his lungs is identified by the numeral 14.

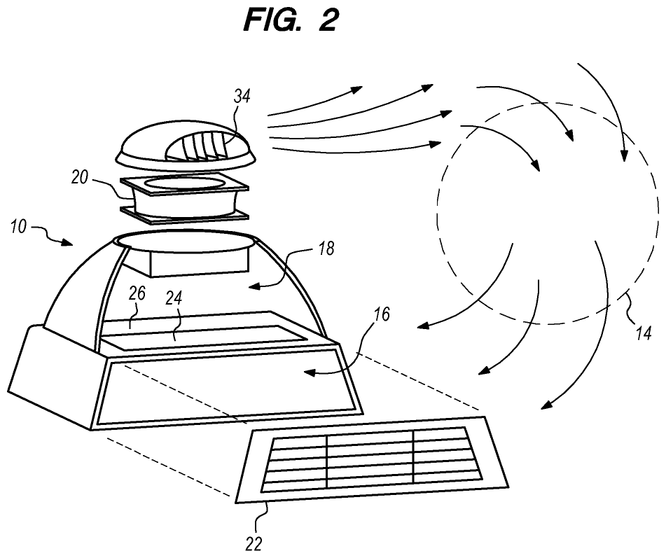

With reference to FIG. 2, the housing of the air sterilizer 10 may be formed from injection molded plastic and conveniently has two compartments, a lower compartment 16 where the sterilization of the air takes place, and an upper compartment 18 which houses the air mover 20. The plastic selected may be any suitable conventional material, desirably one with a very low reflectance in the UV-C band. Polycarbonates have been found to be acceptable when exposed to UV-C radiation over the life expectancy of the germicidal light source 30.

As shown in FIGS. 2-4, air enters the compartment 16 through the input aperture 22, which is conventionally louvered to prevent the line-of-sight escape of UV-C radiation from the compartment 16 into the room. The irradiated air exits the compartment 16 through the opening 24 in the compartment divider 26.

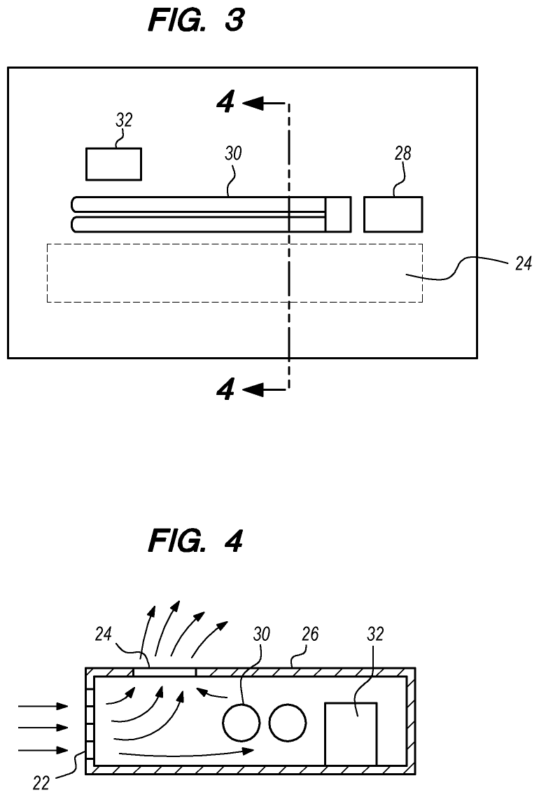

As shown in FIGS. 3 and 4, a suitable conventional source of UV-C radiation is provided in the compartment 16. As illustrated, there is a socket 28 into which a lamp conveniently consisting of a single ended base and two parallel tubes 30 is inserted. The socket 28 is connected through wires (not shown) and a suitable conventional ballast 32. When power is supplied through a conventional cord and switch (not shown), the radiation compartment is filled with UV-C radiation. The position of the lamp 30 relative to the opening 24 in the divider 26 ensures that there is no direct line-of-sight from the lamp 30 to the output aperture 34 that could possibly cause injury to humans.

The position of the lamp 30, and the limited amount of space around it through which the air is forced to flow, serves to control the exposure to the UV-C radiation of the air passing through the chamber 16. The amount of UV-C radiation is desirably adjusted by the combination of UV-C intensity emanating from the lamp 30 and the speed of the air passing through the radiation to kill over 90%, preferably 98%, of common aerosolized pathogens. By way of example, an air mover 20 that circulates air at about 13-20 cfm (dependent on humidity) in combination with a UV-C source 30 that provides at least 500 microwatts per square centimeter of UV-C three inches from the source has proven effective in killing more than 90% of a baseline bacteria drawn through the radiation.

As discussed supra, there is no direct line of sight through any opening in the unit housing to the UV-C lamp 30 and the low reflectance of the material of which the housing is made substantially reduces the likelihood that any UV-C radiation can exit the air sterilizer along an indirect path. It is important for safety reasons that the radiation intensity in the peopled area outside of the zone not exceed the thresholds established by the American Council of Governmental and Industrial Hygiene (ACGIH), i.e., 0.4 microwatts per square centimeter of UV-C irradiance nor exceed total exposure above 6.0 micro joules per square centimeter over an 8 hour period. Tests of the embodiment of FIG. 1 show that exposure to UV-C radiation 6 centimeters from the housing in any direction is less than 0.2 microwatts per square centimeter.

With reference to FIG. 2, the sterilized air from the compartment 16 is drawn upwardly through the opening 24 in the compartment divider 26 under the impetus of the air mover 20, which may be a suitable conventional enclosed fan such as those found in desk top computers. This air is forced upward and out of an output aperture 34 which may be selectively rotatable to vary the direction from which sterilized air is exhausted from the air sterilizer 10.

The output aperture 34 may take any suitable generally “square” cross-section shape such as a square, rectangle, circle or oval. The area of the input aperture is small relative to the area of the input aperture 22 so that the velocity of air expelled is greater than the velocity of the air drawn into the unit. Moreover, the shape of the outlets is desirably elected so that air is drawn from the general direction of the personal breathing space rather than an from an arc around the unit, and expelled along a relatively focused path in the direction of the personal breathing space.

The present invention is preferably powered by plugging it into a conventional electrical receptacle and thus may be used in residential as well as commercial applications.

The air passing through the air sterilizer 10 may be pulled through a suitable conventional 0.25 micron foam particulate filter if filtering is desired in addition to irradiation.

In operation, an individual physically at rest inhales about 15 cubic feet in an hour, all of which is drawn from the personal breathing space of the individual. The air sterilizer of the present invention will sterilize about 25 times that volume (400+ cubic feet) of air. In effect, the air sterilizer provides a constant stream of sterilized air from the relatively small output aperture towards the bubble 14. The sterilized air entering the bubble 14 is mixed with the air between the output aperture 34 and the bubble 14, and with the air already in the bubble, and the mixture inhaled. The inhaled air is substantially germ free because of the high percentage of recently sterilized air.

Exhaled nasal air generally travels downwardly and is mixed with air proximate to the individual in the area 15 which may not have been sterilized, and a portion of that mixture is drawn back to the input aperture 22 where the process is repeated. The input aperture 22 is generally linear facing and draws air principally from the general direction of the personal breathing space rather than from an arc centered on the unit thus increasing the percentage of air that has previously been sterilized.

### Advantages and Scope of Invention

It is to be recognized that the direction to which sterilized air must be directed to reach the bubble 14 will influenced by many factors including air conditioning systems, ceiling fans, the relative temperature of the sterilized air, etc., and that the ability to make minor adjustments in directing the sterilized air are important in ensuring that the personal breathing space remains essentially germ free. To this end, the output aperture 34 of FIG. 2 is desirably sized and shaped to direct sterilized air through a path of shortest distance and minimum obstructions toward the location of the preselected personal breathing space.

The output aperture 34 is relatively small when compared to the input aperture 22 so that the sterilized air may be focused in the direction of the personal breathing space 14. Conversely, the input aperture 22 is relatively large and broadly focused in the direction of the personal breathing space, i.e., the air proximate to the individual in the area generally denoted 15. This increases the likelihood that a portion of the air drawn into the input aperture 22 of the unit 10 will have been previously sterilized, thus further decreasing the concentration of germs in the personal breathing space 14.

The reversal of the direction of air flow through known personal space air sterilizers, i.e., exhaustion from the top rather than the bottom, reduces potential obstructions to the flow of sterilized air to the personal breathing space from items on the desk. Moreover, the heating of the air being sterilized by the UV-C source aids, rather than opposes, the impetus of the air mover increasing the velocity and assisting in the delivery of sterilized air to the personal breathing space.

The horizontal orientation of the UV-C lamp within the personal space air sterilizer lowers the profile, and avoids the deleterious effects of lamp heated air on the cathode of the source prolonging its useful life. More importantly, the horizontal orientation of the lamp facilitates a broad faced, linear input aperture focused on the general direction of exhaled air.

As with known personal space air sterilizers, these germ reduction benefits are cumulative, i.e., as tests have shown with prior art devices, if the number of aerosolized germs such as the influenza virus and SARS is reduced by 98% in one pass through the UV-C killing field, a still greater reduction should be achieved when the air in the first pass through the unit is mixed with the air in the bubble, and the mixture is again passed through the UV-C killing field. A significant reduction in the transmission of germs is expected as the air from the bubble is repeatedly cycled through the UV-C killing field by the focus of the input and output apertures.

By way of example, a food delivery person may shed virus at the doorway of the resident and the suspended germs are ultimately diluted through air exchanges. The resident is exposed to a relatively higher concentration of the virus at the doorway, but only for a short period of time. When the resident returns to his favorite chair where his personal air sterilizer is in use on the adjacent table, the critical personal breathing space, i.e., the bubble surrounding the resident, has been, and will continue to be, immediately sterilized, greatly reducing the likelihood of infection.

While preferred embodiments of the present invention have been described, it is to be understood that the embodiments described are illustrative only and many variations and modifications will naturally occur to those of skill in this art from a perusal hereof. Accordingly, the scope of the invention is to be defined solely by the appended claims when accorded a full range of equivalence.

What is claimed is:

13. A method of immediately protecting a targeted one of a plurality of potential personal breathing spaces within a room having a plurality of potential personal breathing spaces by immediately reducing the concentration of germs within the targeted personal breathing space without immediately reducing the concentration of germs in the plurality of non-targeted personal breathing spaces, the method comprising the steps of:
(a) providing a germ concentration reducing unit having:
(i) an internal passageway substantially impervious to UV-C radiation with a fixed direction input aperture and a selectively variable direction output aperture,
(ii) an air mover for drawing air into the passageway through the input aperture and expelling air from the passageway through the output aperture at a volume that is about twenty five times the air inhaled in an hour from one of the plurality of potential breathing spaces by an individual at rest and less than the volume which would be required to immediately influence the concentration of germs in the room in its entirety, the configuration of the input and output apertures being such that the air expelled has greater speed and is more directionally focused than the air drawn in, and
(iii) a source of UV-C radiation for irradiating the air passing through the passageway;
(b) locating the germ concentration reducing unit in a room sufficiently large to have a plurality of potential personal beathing spaces, the location being proximate to a specific one of the plurality of potential personal breathing spaces to thereby identify that specific one of the potential personal breathing spaces as the targeted personal breathing space to be immediately protected;
(c) orienting the germ concentration reducing unit so that the fixed direction input aperture faces the targeted personal breathing space; and
(d) selectively orientating the selectively variable direction output aperture of the germ concentration reducing unit so that the output aperture focuses expelled air in the direction of the targeted personal breathing space,
whereby air from the general direction of the targeted personal breathing space is drawn into the passageway where it is sterilized and focused to be expelled in the direction of the targeted personal breathing space
thereby immediately reducing the concentration of germs within the targeted personal beathing space without immediately reducing the concentration of germs within the non-targeted personal breathing spaces within the room.

14. The method of claim 13 including the further step of:
selectively adjusting the volume of air circulated by said air mover and the amount of UV-C radiation provided by said source to provide sufficient radiation to kill over 90% of the pathogens in the circulated air in a single pass.

15. In a room sufficiently large to have a plurality of potential personal breathing spaces, apparatus for immediately protecting a targeted one of said plurality of potential personal breathing spaces proximate to aid apparatus by immediately reducing the concentration of germs within said targeted personal breathing space without immediately reducing the concentration of germs in the plurality of non-targeted personal breathing spaces within said room, said apparatus comprising:
an internal passageway substantially impervious to UV-C radiation with a fixed direction input aperture and a selectively variable direction output aperture,
an air mover for drawing air into said passageway through said input aperture and expelling air from said passageway through said output aperture at a volume sufficient to immediately influence the concentration of germs in said targeted personal breathing space but insufficient to immediately influence the concentration of germs in said room in its entirety, the configuration of said input and said output apertures being such that the air expelled has greater speed and is more directionally focused than the air drawn in, and
a source of UV-C radiation for irradiating the air passing through said passageway,
said fixed direction input aperture being oriented to face said targeted personal breathing space so that air drawn into said fixed direction input aperture includes some air from said targeted personal beathing space, and
said selectively variable direction output aperture being selectively oriented to face said targeted personal breathing space so that the output aperture focuses expelled air in the direction of said targeted personal breathing space,
whereby air from the general direction of said targeted personal breathing space is drawn into said passageway where it is sterilized and focused to be expelled in the direction of said targeted personal breathing space
thereby immediately reducing the concentration of germs within said targeted personal beathing space without immediately reducing the concentration of germs within the non-targeted personal breathing spaces within said room.

16. The apparatus of claim 15 wherein said air mover circulates air at about 13-20 cfm and said UV-C source provides at least 500 microwatts of UV-C per square centimeter at a distance of three inches from the source.

17. The apparatus of claim 15 wherein volume of air circulated by said air mover and the amount of UV-C radiation provided by said source are adjustable to provide sufficient radiation to kill over 90% of the pathogens in the circulated air in a single pass.
